(19) United States
(12) Patent Application Publication
(10) Pub. No.: US 2025/0179010 A1
(43) Pub. Date: Jun. 5, 2025

(54) UREA PRODUCTION METHOD AND UREA PRODUCTION APPARATUS

(71) Applicant: Toyo Engineering Corporation, Minato-ku, Tokyo (JP)

(72) Inventors: Takahiro YANAGAWA, Narashino-shi, Chiba (JP); Yasuhiko KOJIMA, Narashino-shi, Chiba (JP); Keiji SANO, Narashino-shi, Chiba (JP); Shogo KAWATA, Narashino-shi, Chiba (JP)

(21) Appl. No.: 18/845,062

(22) PCT Filed: Mar. 16, 2023
(86) PCT No.: PCT/JP2023/010278

§ 371 Date: Sep. 9, 2024

(30) Foreign Application Priority Data
Mar. 16, 2022 (JP) 2022-041381

(51) Int. Cl. C07C 273/10; C25B 1/04

(52) U.S. Cl. CPC C07C 273/10; C25B 1/04

(57) ABSTRACT

Provide are a urea production method and a urea production apparatus in which hydrogen and oxygen are produced by electrolysis of water in an electrolysis unit, nitrogen is separated and recovered from air in an air separation unit, ammonia is synthesized in an ammonia synthesis unit using hydrogen from the electrolysis unit and nitrogen from the air separation unit as raw materials, carbon dioxide is produced by combusting a fuel in an oxycombustion unit while using at least the oxygen from the electrolysis unit, and urea is synthesized in a urea synthesis unit by using the carbon dioxide and the ammonia as raw materials.

### TECHNICAL FIELD

The present invention relates to a urea production method and a urea production apparatus used for producing urea, and more particularly, to a urea production method and a urea production apparatus in which oxygen generated by electrolysis of water is fed into a combustion unit and then carbon dioxide which is the combustion exhaust gas is used as a raw material.

### BACKGROUND ART

In order to produce urea in a urea plant, ammonia and carbon dioxide are required as raw materials. Among these raw materials, it is known that ammonia can be synthesized from nitrogen and hydrogen. Carbon dioxide can be obtained, for example, by combusting a fuel.

Patent Document 1 discloses a method of separating and recovering nitrogen from air and reacting the nitrogen with hydrogen to synthesize ammonia. In addition, it also discloses a method of producing urea by using carbon dioxide and ammonia as raw materials. The carbon dioxide is produced by combusting a fuel by using the gas after nitrogen is separated, that is high concentration oxygen.

### RELATED ART DOCUMENTS

### Patent Documents

Patent Document 1: JP2017-504778

### SUMMARY OF INVENTION

### Technical Problem

However, the method described in Patent Document 1 is a method whose main purpose is simply to improve an oxycombustion system involving synthesis of ammonia, and is not a method whose main purpose is to produce urea. When urea is produced by using ammonia and carbon dioxide as raw materials according to Patent Document 1, since the amount of carbon dioxide produced is small, the excess amount of ammonia becomes very large. Therefore, the method described in Patent Document 1 does not necessarily have a sufficient material balance for producing urea.

Therefore, the object of the present invention is to provide a urea production method and a urea production apparatus with improved material balance.

### Solution to Problem

The present inventors have conducted intensive studies to accomplish the above-mentioned purpose and found that it is very effective to produce urea by using carbon dioxide as a raw material wherein the carbon dioxide is obtained in an oxycombustion step while using oxygen obtained by electrolysis of water, and leading to completion of the present invention.

The present invention is a urea production method comprising
an electrolysis step of producing hydrogen and oxygen by electrolysis of water;
an air separation step of separating and recovering nitrogen from air;
an ammonia synthesis step of synthesizing ammonia by using at least part of the hydrogen produced in the electrolysis step and at least part of the nitrogen separated and recovered in the air separation step as raw materials;
an oxycombustion step of producing carbon dioxide by combusting a fuel while at least using at least part of the oxygen produced in the electrolysis step; and
a urea synthesis step of synthesizing urea by using at least part of the carbon dioxide produced in the oxycombustion step and at least part of the ammonia produced in the ammonia synthesis step as raw materials.

In addition, the present invention is a urea production apparatus having
an electrolysis unit (E) for producing hydrogen and oxygen by electrolysis of water;
an air separation unit (A) for separating and recovering nitrogen from air;
an ammonia synthesis unit (N) for synthesizing ammonia by using at least part of the hydrogen produced in the electrolysis unit (E) and at least part of the nitrogen separated and recovered in the air separation unit (A) as raw materials;
an oxycombustion unit (O) for producing carbon dioxide by combusting a fuel while at least using at least part of the oxygen produced in the electrolysis unit (E); and
a urea synthesis unit (U) for synthesizing urea by using at least part of the carbon dioxide produced in the oxycombustion unit (O) and at least part of the ammonia produced in the ammonia synthesis unit (N) as raw materials.

### Advantageous Effect of Invention

As industrial methods for supplying hydrogen, for example, a steam reforming method using light hydrocarbons (e.g., light naphtha and natural gas) and a catalytic reforming method using heavy naphtha in oil refining are known. On the other hand, an electrolysis method for producing hydrogen by electrolyzing water is also known. This electrolysis method uses water as a raw material and produces hydrogen and oxygen. Oxygen produced by the electrolysis method is then generally released to atmosphere. The present inventors paid attention that it is very effective to use hydrogen as a raw material for ammonia synthesis wherein the hydrogen is obtained by the electrolysis method, and simultaneously utilize the oxygen that has been generally released to atmosphere. That is, in the present invention, the amount of oxygen that can be used in the oxycombustion step is significantly increased because the oxygen obtained by the electrolysis of water is used in the oxycombustion step, and the carbon dioxide obtained in the step is used as a raw material to produce urea. As a result, the excess amount of ammonia is reduced and the material balance for producing urea is improved.

Furthermore, in the present invention, because the amount of oxygen that can be used in the oxycombustion step is significantly increased, the amount of heat obtained in the oxycombustion step is increased so that the amount of steam produced is increased. As a result, the amount of steam required for the urea synthesis step can be sufficiently covered. Additionally, the excess steam can be used as a heat source or used in power generation.

Oxygen obtained by an air separation step, which is used in a general oxycombustion step, contains a small amount of nitrogen and argon. If only oxygen containing the small amount of nitrogen and argon is used in the oxycombustion step, the purity of the carbon dioxide obtained by the oxycombustion step is lowered. As a result, when the obtained carbon dioxide is used for urea synthesis, a step of purifying carbon dioxide may be required. In contrast, in the present invention, the oxygen used in the oxycombustion step contains almost no nitrogen or argon because the oxygen is obtained by electrolysis of water. As a result, a step of purifying carbon dioxide is rarely required, and the processes also can be simplified.

### BRIEF DESCRIPTION OF DRAWINGS

FIG. 1 is a process flow diagram of a urea production apparatus using a urea production in the present invention.

FIG. 2 is a process flow diagram in Example 1.

FIG. 3 is a process flow diagram in Example 2.

FIG. 4 is a process flow diagram in Example 3.

FIG. 5 is a process flow diagram in Comparative Example 1.

### MODES FOR CARRYING OUT THE INVENTION

The reason why the excess amount of ammonia is reduced in the present invention is described below.

First, in the method as described in Patent Document 1, the oxygen used in the oxycombustion step is only oxygen obtained by the air separation step. In the method, because any electrolysis step such as the electrolysis step in the present invention is not conducted, the excess amount of ammonia is increased. For example, when biomass fuel (represented by cellulose C₆H₁₀O₅) is used in the oxycombustion step in the method, the reaction formula is as shown in Formula 1 below.

3H₂+N₂+1/4O₂+1/24C₆H₁₀O₅→2NH₃+1/4CO₂+5/24H₂O→1/4H₂NCONH₂(Urea)+3/2NH₃+11/24H₂O Formula 1

In the left side (raw material) of Formula 1, the ratio of each component is determined based on one equivalent of nitrogen (N₂). For hydrogen (H₂), an equivalent ratio of “3” of theoretical equivalents (H₂:N₂=3:1) for ammonia synthesis is adopted as a ratio. For oxygen (O₂), the composition of the air used in the air separation step is O₂=20.9%, N₂=78.1%, but for simplicity the ratio of oxygen (O₂) to nitrogen (N₂) is expressed as considered as “1/4”, and this is taken as the equivalent.

As shown in the right side of Formula 1 (product), when biomass fuel (cellulose C₆H₁₀O₅) is used, only 1/4 equivalent oxygen of nitrogen is obtained from the air separation step. Therefore, only 1/4 equivalent of carbon dioxide (CO₂) is obtained in the oxycombustion step. In contrast, in the ammonia synthesis step, 2 equivalents of ammonia (NH₃) are produced. Therefore, the equivalence ratio (NH₃/CO₂) is 8.

The theoretical equivalence ratio of ammonia to carbon dioxide for urea synthesis is 2, as shown in Formula 2 below. Therefore, in the method (NH₃/CO₂=8) as described in Patent Document 1, amount of carbon dioxide is insufficient, and a large amount of excess ammonia that cannot be consumed is generated.

2NH₃+CO₂→H₂NCONH₂(Urea)+H₂O Formula 2

In contrast, when the oxygen obtained by the electrolysis step is used in the oxycombustion step as in the present invention, the excess amount of ammonia is reduced. For example, when biomass fuel (represented by cellulose C₆H₁₀O₅) is used in the oxycombustion step in the method of the present invention, the reaction formula is as shown in Formula 3 below.

3H₂+N₂+3/2O₂+1/6C₆H₁₀O₅→2NH₃+CO₂+5/6H₂O+1/2O₂→H₂NCONH₂(Urea)+11/6H₂O+1/2O₂ Formula 3

In the left side (raw material) of Formula 3, the ratio of each component is determined based on one equivalent of nitrogen (N₂). For hydrogen (H₂), an equivalent ratio of “3” of theoretical equivalents (H₂:N₂=3:1) for ammonia synthesis is adopted as a ratio. For oxygen (O₂), production ratio (H₂:O₂=2:1) in electrolysis of water (H₂O) is taken as a base, and an equivalent ratio of “3/2” is adopted as the ratio which is an amount of oxygen (O₂) produced when obtaining 3 equivalents of hydrogen (H₂).

As shown in the right side of Formula 3 (product), when biomass fuel (cellulose C₆H₁₀O₅) is used in the present invention, 3/2 equivalent of oxygen is obtained in the electrolysis step, then 1 equivalent of carbon dioxide is obtained in the oxycombustion step, and subsequently 2 equivalents of ammonia (NH₃) are obtained in the ammonia synthesis step. Therefore, the equivalent ratio (NH₃/CO₂) is 2. The theoretical equivalent for urea synthesis is 2 as mentioned above. In conclusion, in the method of the present invention in this case, there is no shortage of carbon dioxide and no excess ammonia is generated.

Next, the case of using a fossil fuel (represented by methane CH₄) in the oxycombustion step in the method of the present invention is described. The reaction formula in the case is as shown in Formula 4 below.

3H₂+N₂+3/2O₂+3/4CH₄→2NH₃+3/4CO₂+3/2H₂O→3/4H₂NCONH₂(Urea)+1/4NH₃+5/2H₂O Formula 4

The equivalence ratios of nitrogen (N₂), hydrogen (H₂) and oxygen (O₂) on the left side (raw material) of Formula 4 are the same as in Formula 3.

As shown in the right side of Formula 4 (product), when a fossil fuel (methane CH₄) is used in the present invention, 3/2 equivalent of oxygen is obtained in the electrolysis step, then 3/4 equivalent of carbon dioxide (CO₂) is obtained in the oxycombustion step and subsequently 2 equivalents of ammonia (NH₃) are obtained in the ammonia synthesis step. Therefore, the equivalent ratio (NH₃/CO₂) is 8/3=about 2.7. On the other hand, the theoretical equivalent for urea synthesis is 2 as described above. Therefore, in the method of the present invention in this case, carbon dioxide is slightly deficient, and excess ammonia is slightly generated. However, compared with the method (NH₃/CO₂=8) described as in Patent Document 1, it can be said that the excess amount of ammonia is significantly reduced.

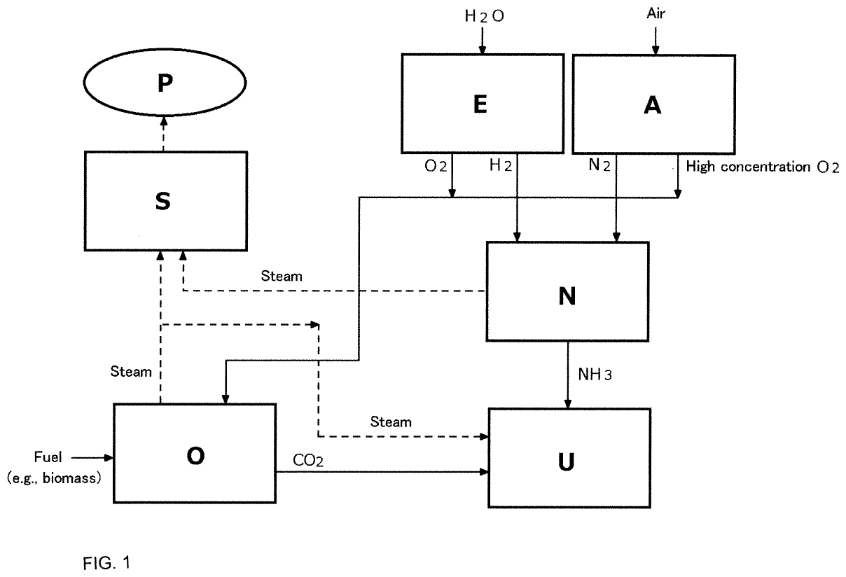

FIG. 1 is a process flow diagram of a urea production apparatus using a urea production in the present invention. Each step and each unit are explained below.

### [Electrolysis Unit (E)]

The electrolysis unit (E) shown in FIG. 1 is a unit for conducting the electrolysis step of producing hydrogen and oxygen by electrolysis of water. Regarding the specific electrolysis conditions in the electrolysis step and the structures of the electrolysis unit (E), known conditions and known structures for electrolysis of water can be employed without limitation.

As shown in FIG. 1, water is fed into the electrolysis unit (E). Then, hydrogen and oxygen are produced by electrolysis of this water. At least part of the obtained hydrogen is fed into the ammonia synthesis unit (N) explained later, and it is used as a raw material for synthesizing ammonia. On the other hand, at least part of the obtained oxygen is fed into the oxycombustion unit (O) explained later, and it is used as a raw material for producing carbon dioxide.

Electric power is required to conduct the electrolysis step. The type of the used electric power is not particularly limited. However, it is preferable in environmental protection to use electric power generated by using renewable energy. Renewable energy is energy that always exists in natural world, such as biomass fuel, sun light, wind power, geothermal and water power. For example, a power generation using combustion heat obtained by combusting the biomass fuel is excellent in carbon neutral. In addition, a power generation by sun light, wind power or geothermal it is excellent in that no carbon dioxide is emitted through the power generation. In the present invention, it is also possible that, for example, a biomass fuel is used as a fuel in the oxycombustion unit (O) explained later, and the combustion heat thereof is used to generate electric power, and then at least part of the obtained electric power is used as power for the electrolysis step.

### [Air Separation Unit (A)]

The air separation unit (A) shown in FIG. 1 is a unit for conducting the air separation step of separating and recovering nitrogen from air. Further, the air separation step may be a step of separating and recovers nitrogen from air and producing a gas containing a high concentration oxygen. Regarding the specific separation conditions in this air separation step and the structures of the air separation unit (A), known conditions and known structures for air separation can be employed without limitation. Specific examples include cryogenic separation and pressure swing adsorption (PSA).

As shown in FIG. 1, air is fed into the air separation unit (A). Then, nitrogen is separated and recovered from this air to produce a gas containing oxygen in high concentration (the oxygen concentration in the gas is usually 90 to 100% by volume, hereinafter referred to as “high concentration oxygen”). At least part of the separated and recovered nitrogen is fed into the ammonia synthesis unit (N) explained later, and it is used as a raw material for synthesizing ammonia. On the other hand, at least part of the obtained high concentration oxygen is fed into the oxycombustion unit (O) explained later, and it is used as a raw material for producing carbon dioxide.

Although the high concentration oxygen produced in the air separation unit (A) is fed into the oxycombustion unit (O) in FIG. 1, the present invention is not limited to this case. For example, the high concentration oxygen produced in the air separation unit (A) may not be fed into the oxycombustion unit (O), if the necessary amount of carbon dioxide can be sufficiently produced by feeding only the oxygen produced in the electrolysis unit (E) for the oxycombustion unit (O). In this case, it may be for example collected outside the system. Or the structure of the air separation unit (A) may be simplified by employing such structure that nitrogen is separated and recovered from air but without producing high concentration oxygen.

### [Ammonia Synthesis Unit (N)]

The ammonia synthesis unit (N) shown in FIG. 1 is a unit for conducting the ammonia synthesis step of synthesizing ammonia by using at least part of the hydrogen produced in the electrolysis step and at least part of the nitrogen separated and recovered in the air separation step as raw materials. Regarding the specific synthesis conditions in the ammonia synthesis step and the structures of the ammonia synthesis unit (N), known conditions and known structures for ammonia synthesis can be employed without limitation. Specific examples include the Haber process and other industrial ammonia synthesis processes.

As shown in FIG. 1, into the ammonia synthesis unit (N), hydrogen is fed from the electrolysis unit (E), and nitrogen is fed from the air separation unit (A). Then, ammonia is synthesized by using this hydrogen and the nitrogen as raw materials. At least part of the obtained ammonia is fed into the urea synthesis unit (U) explained later, and it is used as a raw material for producing urea.

### [Oxycombustion Unit (O)]

The oxycombustion unit (O) shown in FIG. 1 is a unit for conducting the oxycombustion step of producing carbon dioxide by combusting a fuel while at least using at least part of the oxygen produced in the electrolysis step. Regarding the specific combustion conditions in the oxycombustion step and the structures of the oxycombustion unit (O), known conditions and known structures for carbon dioxide production by combustion can be employed without limitation.

The embodiment shown in FIG. 1 is an embodiment in which carbon dioxide is produced by combusting a fuel while using at least part of the oxygen produced in the electrolysis step and at least part of the high concentration oxygen produced in the air separation step. Such embodiment is particularly preferable in case that only the feed amount of oxygen produced in the electrolysis step is insufficient, such as the case explained above using Formula 4 (the case that fossil fuel is used). However, the present invention is not limited to this embodiment. It is not necessary to feed high concentration oxygen from the air separation unit (A), for example, in case that feed amount of oxygen produced in the electrolysis step is sufficient, such as the case explained above using Formula 3 (the case that biomass fuel is used).

As shown in FIG. 1, into the oxycombustion unit (O), oxygen is fed from the electrolysis unit (E) and high concentration oxygen is fed from the air separation unit (A). Then, the oxygen and the high concentration oxygen are used to combust a fuel to produce carbon dioxide. At least part of the obtained carbon dioxide is fed into the urea synthesis unit (U) explained later and it is used as a raw material for producing urea.

The type of fuel used in the oxycombustion step is not particularly limited. Specific examples thereof include biomass fuels such as wood pellets, organic wastes such as municipal garbage, and fossil fuels such as natural gas, petroleum and coal. Especially, biomass fuels are preferred in carbon neutral compared to fossil fuels.

### [Urea Synthesis Unit (U)]

The urea synthesis unit (U) shown in FIG. 1 is a unit for conducting the urea synthesis step of synthesizing urea by using at least part of the carbon dioxide produced in the oxycombustion step and at least part of the ammonia produced in the ammonia synthesis step as raw materials. Regarding the specific synthesis conditions in the urea synthesis step and the structures of the urea synthesis unit (U), known conditions and known structures for urea synthesis can be employed without limitation.

As shown in FIG. 1, into the urea synthesis unit (U), carbon dioxide is fed from the oxycombustion unit (O) and ammonia is fed from the ammonia synthesis unit (N). Then, the carbon dioxide and the ammonia are used as raw materials for producing urea.

### [Power Generation Unit (S)]

The power generation unit (S) is a power generation unit for conducting the power generation step which is a power generation step of generating electric power by using at least part of thermal energy generated by combustion in the oxycombustion step, and/or a power generation step of generating electric power by using at least part of steam produced using at least part of thermal energy generated by combustion in the oxycombustion step. Regarding the specific power generation conditions in the power generation step and the structures of the power generation unit (S), known conditions and known structures for power generation can be employed without limitation.

In the embodiment shown in FIG. 1, the power generation unit (S) is a power generation unit for conducting a steam turbine power generation step by using a steam for power generation. As shown in FIG. 1, into the power generation unit (S), steams are fed from the oxycombustion unit (O) and the ammonia synthesis unit (N). Then, the steams are used for turbine power generation. The obtained electric power (P) can also be used, for example, in one or more steps selected from the group consisting of the urea synthesis step, the ammonia synthesis step and the electrolysis step.

The embodiment having a steam turbine power generation unit as the power generation unit (S) as shown in FIG. 1 is a preferred embodiment in the present invention. In this embodiment, the steam includes steam produced using at least part of the heat generated by combustion in the oxycombustion unit (O). Since a relatively large amount of oxygen is fed into the oxycombustion unit (O) shown in FIG. 1, the amount of heat generated by combustion is also large. As a result, even if steam is fed in to the urea synthesis unit (U), the excess steam can be used for the power generation.

In addition, in the embodiment shown in FIG. 1, the steam contains not only steam produced by combustion while using at least part of the heat generated in the oxycombustion unit (O), but also steam produced by at least part of heat of reaction in ammonia synthesis (e.g., steam produced by using heat exchanger for cooling and condensing the synthesized ammonia gas). Therefore, the amount of power generation is further increased.

The present invention may have other facilities in addition to the units described above. Other facilities include, for example, heat exchanger for producing steam from combustion heat, and carbon dioxide purification unit (e.g., unit for dehydration and removal of impurities).

The present invention is not limited to the embodiment shown in FIG. 1 explained above. For example, in the embodiment shown in FIG. 1, a steam turbine power generation unit was used as the power generation unit (S). However, other power generation units may be added to it, and steam turbine power generation units may be replaced by another power generation unit. The other power generation unit is, for example, a unit for conducting the power generation step in which at least part of the thermal energy generated by combustion in the oxycombustion step is used directly to generate power. Specific examples thereof include a unit for conducting a gas turbine power generation step and a unit for conducting a supercritical CO₂ cycle power generation process.

### EXAMPLES

Hereinafter, the present invention will be described in more detail with reference to examples. However, the present invention is not limited by the examples.

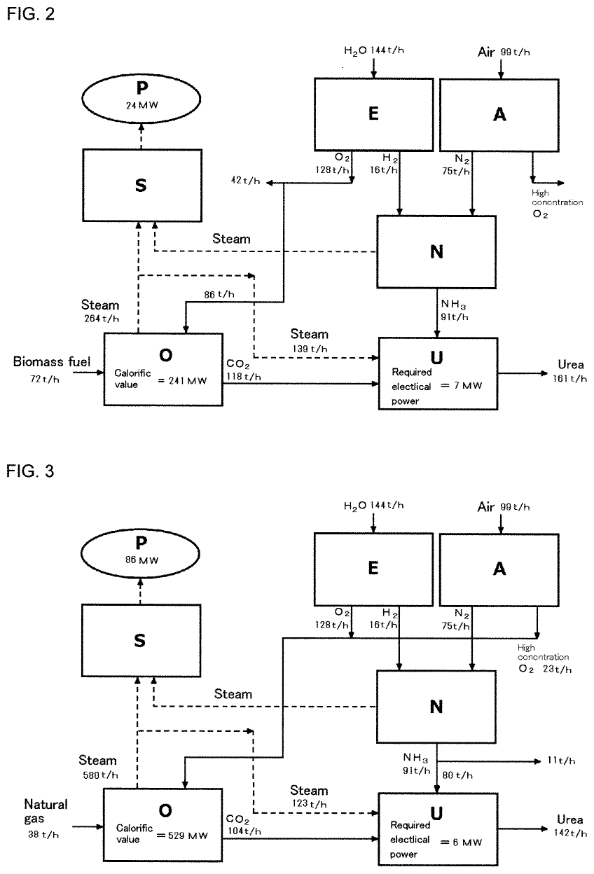

### Example 1 (FIG. 2)

In the electrolysis unit (E), 16 t/h of hydrogen and 128 t/h of oxygen were produced by electrolysis from 144 t/h of water. On the other hand, in the air separation unit (A), 75 t/h of nitrogen was separated and recovered from 99 t/h of air. Then, by using 16 t/h of the hydrogen and 75 t/h of the nitrogen as raw materials, 91 t/h of ammonia was synthesized in the ammonia synthesis unit (N).

86 t/h of oxygen among 128 t/h of the oxygen produced in the electrolysis unit (E) and 72 t/h of biomass as fuel were fed into the oxycombustion unit (O), and then the biomass (wood pellets) was combusted. Then, the generated combustion gas was cooled and subjected to a necessary treatment such as cleaning to obtain 118 t/h of carbon dioxide. The remaining oxygen of 42 t/h was collected outside the system.

118 t/h of carbon dioxide obtained as described above and 91 t/h of ammonia produced in the ammonia synthesis unit (N) were fed into the urea synthesis unit (U) to produce 161 t/h of urea.

The heat of combustion of the biomass was 2,873 kcal/kg, and the amount of heat generation in the oxycombustion unit (O) was about 241 MW. This heat was recovered to produce 264 t/h of steam. 139 t/h of steam among 264 t/h of the steam was fed into the urea synthesis unit (U) and used therein. The remaining steam was fed into the steam turbine power generation unit (S). Furthermore, the steam produced by heat recovered from the ammonia synthesis unit (N) was also fed into the steam turbine power generation unit (S). These steams were used for power generation to obtain 24 MW of electric power. Part of the electric power was supplied into the urea synthesis unit (U) to cover the required power of 7 MW.

### Example 2 (FIG. 3)

In the electrolysis unit (E), 16 t/h of hydrogen and 128 t/h of oxygen were produced by electrolysis from 144 t/h of water. On the other hand, in the air separation unit (A), 75 t/h of nitrogen was separated and recovered from 99 t/h of air to produce 23 t/h of gas containing oxygen in high concentration (oxygen concentration: about 98%). Then, by using 16 t/h of the hydrogen and 75 t/h of the nitrogen as raw materials, 91 t/h of ammonia was synthesized in the ammonia synthesis unit (N).

128 t/h of the oxygen produced in the electrolysis unit (E) and 23 t/h of the gas containing oxygen in high concentration produced in the air separation unit (A) (151 t/h in total) and 38 t/h of natural gas as fuel were fed into the oxycombustion unit (O), and then the natural gas was combusted. Then, the generated combustion gas was cooled and subjected to a necessary treatment such as washing to obtain 104 t/h of carbon dioxide.

104 t/h of the carbon dioxide obtained as described above and 80 t/h of ammonia among 91 t/h of the ammonia produced in the ammonia synthesis unit (N) were fed into the urea synthesis unit (U) to produce 142 t/h of urea. The remaining 11 t/h of ammonia was collected outside the system.

The heat of combustion of the natural gas was 11,950 kcal/kg and the amount of heat generation in the oxycombustion unit (O) was about 529 MW. This heat was recovered to produce 580 t/h of steam. 123 t/h of steam among 580 t/h of the steam was fed into the urea synthesis unit (U) and used therein. The remaining steam was fed into the steam turbine power generation unit (S). Furthermore, the steam produced by heat recovered from the ammonia synthesis unit (N) was also fed into the steam turbine power generation unit (S). These steams were used for power generation to obtain 86 MW of electric power. Part of the electric power was supplied to the urea synthesis unit (U) to cover the required power of 6 MW.

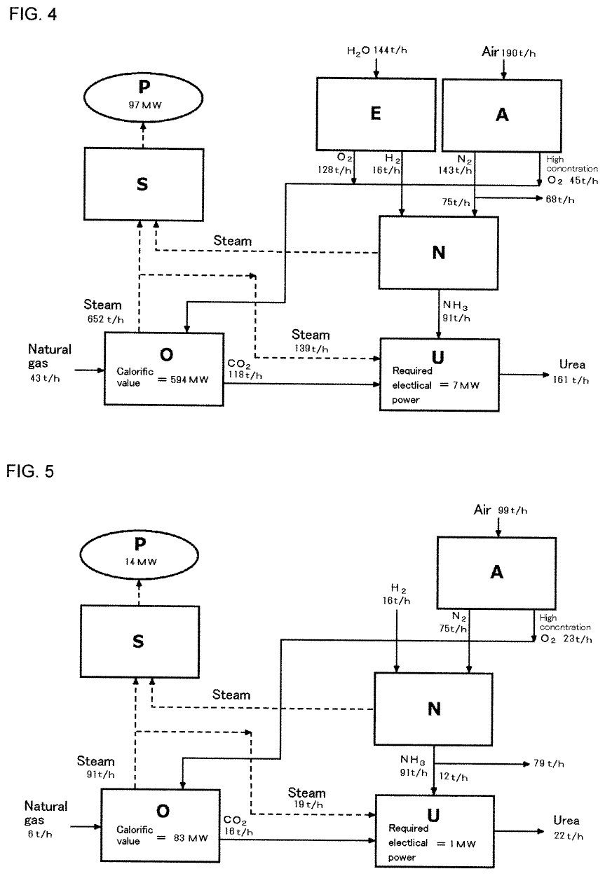

### Example 3 (FIG. 4)

In the electrolysis unit (E), 16 t/h of hydrogen and 128 t/h of oxygen were produced by electrolysis from 144 t/h of water. On the other hand, in the air separation unit (A), 143 t/h of nitrogen was separated and recovered from 190 t/h of air to produce 45 t/h of gas containing oxygen in high concentration (oxygen concentration: about 98%). Then, by using 16 t/h of the hydrogen and 75 t/h of nitrogen among 143 t/h of the nitrogen as raw materials, 91 t/h of ammonia was synthesized in the ammonia synthesis unit (N). The remaining 68 t/h of nitrogen was recovered out of the system.

128 t/h of oxygen produced in the electrolysis unit (E) and 45 t/h of the gas containing oxygen in high concentration produced in the air separation unit (A) (173 t/h in total), 43 t/h of natural gas as fuel were fed into the oxycombustion unit (O) and then the natural gas was combusted. Then, the generated combustion gas was cooled and subjected to a necessary treatment such as washing to obtain 118 t/h of carbon dioxide.

118 t/h of the carbon dioxide obtained as described above and 91 t/h of the ammonia produced in the ammonia synthesis unit (N) were fed into the urea synthesis unit (U) to produce 161 t/h of urea.

The heat of combustion of the natural gas was 11,950 kcal/kg and the amount of heat generation in the oxyfuel unit (O) was about 594 MW. This heat was recovered to produce 652 t/h of steam. 139 t/h of steam among 652 t/h of the steam was fed into the urea synthesis unit (U) and used therein. The remaining steam was fed into the steam turbine power generation unit (S). Furthermore, the steam produced by heat recovered from the ammonia synthesis unit (N) was also fed into the steam turbine power generation unit (S). These steams were used for power generation to obtain 97 MW of electric power. Part of the electric power was supplied to the urea synthesis unit (U) to cover the required power of 7 MW.

### Comparative Example 1 (FIG. 5)

In the air separation unit (A), 75 t/h of nitrogen was separated and recovered from 99 t/h of air to produce 23 t/h of gas containing oxygen in high concentration (oxygen concentration of about 98%). Then, by using 16 t/h of hydrogen separately prepared and 75 t/h of the nitrogen recovered in the air separation unit (A) as raw materials, 91 t/h of ammonia was synthesized in the ammonia synthesis unit (N).

23 t/h of the gas containing oxygen in high concentration produced in the air separation unit (A) and 6 t/h of natural gas as fuel were fed into the oxycombustion unit (O), and then the natural gas was combusted. Then, the generated combustion gas was cooled and subjected to a necessary treatment such as washing to obtain 16 t/h of carbon dioxide.

16 t/h of the carbon dioxide obtained as described above and 12 t/h of ammonia among 91 t/h of the ammonia produced in the ammonia synthesis unit (N) were fed into the urea synthesis unit (U) to produce 22 t/h of urea. The remaining 79 t/h of the ammonia was recovered out of the system.

The heat of combustion of the natural gas was 11,950 kcal/kg, and the amount of heat generation in the oxyfuel unit (O) was about 83 MW. The heat was recovered to produce 91 t/h of steam. 19 t/h of steam among 91 t/h of the steam was fed into the urea synthesis unit (U) and used therein. The remaining steam was fed into the steam turbine power generation unit (S). Furthermore, the steam produced by heat recovered from the ammonia synthesis unit (N) was also fed into the steam turbine power generation unit (S). These steams were used for power generation to obtain 14 MW of electric power. Part of the electric power was supplied to the urea synthesis unit (U) to cover the required power of 1 MW.

Table 1 summarizes the results of the above Examples and Comparative Examples. The unit of raw material in Table 1 represents raw material consumption per production amount of urea (t/t-urea). However, regarding nitrogen and oxygen, those amounts is calculated as intermediate production amount per production amount of urea.

TABLE 1

| | | | | | Comp. |
|---|---|---|---|---|---|
| | | Ex. 1 | Ex. 2 | Ex. 3 | Ex. 1 |
| Production amount of | | | | | |
| final product | | | | | |
| Urea | t/h | 161 | 142 | 161 | 22 |
| NH₃ | t/h | | 11 | | 79 |
| Intermediate production | | | | | |
| amount | | | | | |
| CO₂ | t/h | 118 | 104 | 118 | 16 |
| O₂ (note 1) | t/h | 128 | 151 | 173 | 23 |
| NH₃ | t/h | 91 | 91 | 91 | 91 |
| H₂ | t/h | 16 | 16 | 16 | |
| N₂ | t/h | 75 | 75 | 143 | 75 |
| Amount of supply | | | | | |
| (consumption) | | | | | |
| CO₂ for (U) | t/h | 118 | 104 | 118 | 16 |
| Fuel | t/h | 72 | 38 | 43 | 6 |
| O₂ \|for (O) (note 1) | t/h | 86 | 151 | 173 | 23 |
| NH₃ for (U) | t/h | 91 | 80 | 91 | 12 |
| H₂ for (N) | t/h | 16 | 16 | 16 | 16 |
| N₂ for (N) | t/h | 75 | 75 | 75 | 75 |
| Unit of raw material | | | | | |
| (per production amount of urea) | | | | | |
| N₂ (note 2) | t/t-urea | 0.47 | 0.53 | 0.89 | 3.41 |
| H₂ | t/t-urea | 0.10 | 0.11 | 0.10 | 0.73 |
| Fuel | t/t-urea | 0.45 | 0.27 | 0.27 | 0.27 |
| O₂ (note 2) | t/t-urea | 0.80 | 1.06 | 1.07 | 1.05 |
| Amount of heat generation | MW | 241 | 529 | 594 | 83 |
| Amount of steam generated at (O) | t/h | 264 | 580 | 652 | 91 |
| Amount of electric power generated at (S) | MW | 24 | 86 | 97 | 14 |

(note 1):
O₂ + high concentration O₂
(note 2):
Intermediate production amount basis

As shown in Table 1, the material balances in Examples 1 to 3 were excellent because the electrolysis step (and the steam turbine power generation step) was conducted. In contrast, the material balance in Comparative Example 1 was inferior to that in Examples 1 to 3 because the electrolysis step was not conducted. Specifically, it was as follows.

Example 1 is an example using biomass as a fuel. In Example 1, all the oxygen required for the oxycombustion unit (O) could be supplied by the oxygen produced in the electrolysis unit (E) only. Therefore, the structure of the air separation unit (A) could be simplified, and the entire amount of the ammonia produced could be used for urea synthesis, thereby reducing the generation of excess ammonia to zero. As a result, the minimum values of 0.47 and 0.10 for the units of raw material of nitrogen and hydrogen among the four examples could be achieved. In addition, the heat generated by, for example, combustion was effectively used to generate steam. Therefore, Example 1 is efficient from the viewpoint of urea production, and is very excellent in terms of equipment configuration and material balance.

Example 2 is an example using natural gas as a fuel. When using natural gas as a fuel, more oxygen is required for oxycombustion than when using biomass. Therefore, in Example 2, the oxygen produced in the electrolysis unit (E) and the air separation unit (A) were fed together to the oxycombustion unit (O). However, the amount of oxygen was still insufficient, that is, the produced carbon dioxide was insufficient, and the urea production amount was reduced compared to Example 1 (urea production amount in Example 2=142 t/h, urea production amount in Example 1=160 t/h), and 11 t/h of excess ammonia was produced. As a result, the units of raw material of nitrogen and hydrogen were 0.53 and 0.11 respectively. However, Example 2 is superior to Comparative Example 1 in terms of material balance for urea production. In addition, excess ammonia can be shipped as a by-product.

Example 3 is an example using natural gas as a fuel in which the amount of oxygen produced was increased in order to achieve the same urea production amount (160 t/h) as in Example 1. Specifically, in Example 3, an air separation unit (A) having about twice the production capacity of the air separation unit (A) used in Examples 1 and 2 was used. As a result, the generation of excess ammonia could be eliminated as in Example 1. The units of raw material of nitrogen and hydrogen were 0.89 and 0.10 respectively. In addition, the generated amount of steam and the amount of electric power obtained in the steam turbine power generation unit (S) were greater than those of Examples 1 and 2. However, Example 1 was superior in reducing the capacity of the air separation unit (A) to this example.

Comparative Example 1 is an example in which the electrolysis unit (E) was not used. In Comparative Example 1, the amount of oxygen was small, and the amount of carbon dioxide obtained in the oxycombustion unit (O) was small because oxygen was used only from the air separation unit (A). As a result, the urea production amount was as low as 22 t/h, and 78 t/h of excess ammonia was generated. The units of raw material of nitrogen and hydrogen were 3.41 and 0.73 respectively, which were the worst among the four examples.

### INDUSTRIAL APPLICABILITY

According to the present invention, the material balance in producing urea is improved so that it is very useful as an industrial urea production method and urea production apparatus.

### Explanation of Numerals

P: Electric power
S: Power generation unit
O: Oxycombustion unit
E: Electrolysis unit
A: Air separation unit
N: Ammonia synthesis unit
U: Urea synthesis unit

1. A urea production method with a reduced excess amount of ammonia and improved material balance comprising
an electrolysis step of producing hydrogen and oxygen by electrolysis of water;
an air separation step of separating and recovering nitrogen from air and producing gas containing high concentration oxygen;
an ammonia synthesis step of synthesizing ammonia by using at least part of the hydrogen produced in the electrolysis step and at least part of the nitrogen separated and recovered in the air separation step as raw materials;
an oxycombustion step of producing carbon dioxide by combusting a fuel while at least using at least part of the oxygen produced in the electrolysis step and at least part of the gas containing high concentration oxygen produced in the air separation step; and
a urea synthesis step of synthesizing urea by using at least part of the carbon dioxide produced in the oxycombustion step and at least part of the ammonia produced in the ammonia synthesis step as raw materials.

2. (canceled)

3. The urea production method according to claim 1, wherein the urea production method comprises a power generation step, and the power generation step is
a power generation step of generating electric power by using at least part of thermal energy generated by combustion in the oxycombustion step, and/or
a power generation step of generating electric power by using at least part of steam produced using at least part of thermal energy generated by combustion in the oxycombustion step.

4. The urea production method according to claim 3, wherein at least part of the electric power generated in the power generation step is used in at least one step selected from the group consisting of the urea synthesis step, the ammonia synthesis step and the electrolysis step.

5. The urea production method according to claim 3, wherein the power generation step is a power generation step of generating electric power by using
steam produced using at least part of thermal energy generated by combustion in the oxycombustion step, and
steam produced using at least part of heat of reaction from ammonia synthesis in the ammonia synthesis step.

6. A urea production apparatus with a reduced excess amount of ammonia and improved material balance comprising
an electrolysis unit (E) for producing hydrogen and oxygen by electrolysis of water;
an air separation unit (A) for separating and recovering nitrogen from air and producing gas containing high concentration oxygen;
an ammonia synthesis unit (N) for synthesizing ammonia by using at least part of the hydrogen produced in the electrolysis unit (E) and at least part of the nitrogen separated and recovered in the air separation unit (A) as raw materials;
an oxycombustion unit (O) for producing carbon dioxide by combusting a fuel while at least using at least part of the oxygen produced in the electrolysis unit (E) and at aleast part of the gas containing high concentration oxygen produced in the air separation unit (A) as raw materials; and
a urea synthesis unit (U) for synthesizing urea by using at least part of the carbon dioxide produced in the oxycombustion unit (O) and at least part of the ammonia produced in the ammonia synthesis unit (N) as raw materials.

7. (canceled)

8. The urea production apparatus according to claim 6, wherein the urea production apparatus comprises a power generation unit (S), and the power generation unit (S) is
a power generation unit for generating electric power by using at least part of thermal energy generated by combustion in the oxycombustion unit (O), and/or
a power generation unit for generating electric power by using at least part of steam produced using at least part of thermal energy generated by combustion in the oxycombustion unit (O).

9. The urea production apparatus according to claim 8, wherein at least part of the electric power generated in the power generation unit (S) is used in at least one unit selected from the group consisting of the urea synthesis unit (U), the ammonia synthesis unit (N) and the electrolysis unit (E).

10. The urea production apparatus according to claim 8, wherein the power generation unit (S) is a power generation unit for generating electric power by using
steam produced using at least part of thermal energy generated by combustion in the oxycombustion unit (O), and
steam produced using at least part of heat of reaction from ammonia synthesis in the ammonia synthesis unit (N).
